(19) United States
(12) Patent Application Publication
(10) Pub. No.: US 2019/0189271 A1
(43) Pub. Date: Jun. 20, 2019

(54) CONTAINER TRACKING SYSTEM

(71) Applicant: Clinical Biotechnology Research Institute at RSFH, Charleston, SC (US)

(72) Inventors: Jacobo E. Mintzer, Sullivans Island, SC (US); Ashley Severance, Charleston, SC (US); Andrea H. Marshall, Mt. Pleasant, SC (US); Krista Herndon, Summerville, SC (US)

(73) Assignee: Clinical Biotechnology Research Institute at RSFH, Charleston, SC (US)

(21) Appl. No.: 16/318,891

(22) PCT Filed: Jul. 20, 2017
(86) PCT No.: PCT/US2017/042967

§ 371 Date: Jan. 18, 2019

Related U.S. Application Data
(60) Provisional application No. 62/365,131, filed on Jul. 21, 2016.

(51) Int. Cl. G16H 40/20; G01D 21/02

(52) U.S. Cl. CPC G16H 40/20; G01D 21/02

(57) ABSTRACT

Devices and methods protect a recipient receiving a medical fluid from a donor such as expressed breast milk from unsafe handling, expiration, and misidentification. For example, each container employs a container tracking device to measure one or more of temperature, duration, and patient identity, and receives a permission code from a receiving computer based thereon. The permission code directs the container tracking device to provide an appropriate alert, thereby informing medical personnel whether to administer the medical fluid from a given container to the recipient. Optionally, a medical fluid supply can be managed by identifying those containers having the shortest remaining shelf life, in certain cases.

### CROSS REFERENCE TO RELATED APPLICATIONS

The present application claims benefit of priority under PCT Article 8 and 35 U.S.C. 119(e) of U.S. Provisional Patent Application No. 62/365,131, filed on Jul. 21, 2016, entitled, “CONTAINER TRACKING SYSTEM.” That provisional patent application is incorporated herein by reference in its entirety.

### FIELD OF INVENTION

The present invention relates to devices and methods for handling a medical fluid.

### BACKGROUND OF THE INVENTION

In certain settings, it is necessary to ensure proper handling and management of fluids stored in containers to protect the integrity of the contents of the containers. For example, breast milk that has been expressed must be identified and stored in a controlled environment until it is administered to the mother's infant. This could apply to a clinical setting such as a hospital nursery, as well a non-clinical setting such as a daycare. In another example, intravenous (IV) fluid used to prevent or correct problems with a patient's fluid status must be monitored to eliminate administering the IV fluid to the wrong patient, or issuing expired stock. In still another example, the administration of certain medications that require preparation in pharmacy settings needs to be managed under appropriate environmental and engineering controls to ensure storage conditions are met.

The storage and handling of expressed breast milk will be used to illustrate one device of the invention and its use. When mothers and babies are separated in the hospital or daycare environment, the risk of expressed breast milk mismanagement increases. A common misidentification error in the nursery involves feeding a mother's expressed breast milk to the wrong infant. The factors identified in these events included incorrectly labeled containers, difficult-to-read handwritten container labels, errors in verification of patient/container correlation, and systematic problems with the storage of expressed breast milk containers.

Ensuring consistent delivery of the correct mother's expressed breast milk to the correct baby is a challenge. When an infant is fed a different mother's expressed breast milk, both the family and staff experience anxiety regarding the potential risk to the infant, and uncertainty regarding how to respond to exposure when an error occurs. In some hospital settings an incident of delivering mismatched breastmilk to an infant triggers procedural guidelines recommending extensive testing of the infant that is not only costly but also stressful.

Another risk includes failure to monitor refrigerator and container temperatures, as well as the age of the milk. Verification that the bottle contents have been stored and handled within acceptable temperature ranges is critical to maintaining the nutritional value and safety of the expressed breast milk. Tracking shelf life and usage rate of the bottles to ensure the oldest milk is used first, and that expired milk is discarded once it is past an acceptable age, ensures the milk administered to the infant is used within an acceptable timeframe.

### SUMMARY OF THE INVENTION

Applicants have invented devices and methods for a fully-functioning tracking system for expressed breast milk or other medical fluids. Some embodiments of the present invention provide a device to track a container, one such device comprising: a temperature sensor configured to measure an exterior temperature of a container that correlates to a temperature of a fluid in the interior of the container; a proximity sensor configured to interrogate an identity of a donor or potential recipient when the container tracking device and the donor or potential recipient are brought in close proximity; a timer configured to provide time data; a processor which is communicatively coupled to the temperature sensor, the proximity sensor and the timer, the processor being configured to receive temperature data from the temperature sensor, identity data from the proximity sensor, and time data from the timer; a transmitter communicatively coupled to the processor, the transmitter configured to send temperature data, identity data, and time data to a receiving computer; a receiver communicatively coupled to the processor, the receiver configured to receive a permission code from the receiving computer; an alert system communicatively coupled to the processor, the alert system configured to provide an alert based on the permission code; and a power supply.

Further embodiments of the present invention relate to a system for tracking a plurality of containers, comprising: the plurality of containers, wherein each container is adapted to receive, store, and dispense a fluid; a plurality of container tracking devices, each container tracking device in the plurality of container tracking devices being coupled to a container in the plurality of containers, each container tracking device being configured to measure an exterior temperature of the container that correlates to a temperature of a fluid in the interior of the container, a proximity sensor configured to interrogate an identity of a donor or potential recipient when the container tracking device and the donor or potential recipient are brought in close proximity, and to time a duration of the fluid in each container to obtain time data. Each container tracking device is further configured to selectively receive permission codes, and provide alerts based on the permission codes.

Still other embodiments relate to an identifying device for a donor configured to be interrogated by the proximity sensor of each container tracking device in the plurality of container tracking devices; optionally, an identifying device for an intended recipient, wherein in some cases the donor is related to the recipient, the identifying device for the recipient being configured to be interrogated by the proximity sensor of each container tracking device in the plurality of container tracking devices.

Still further embodiments comprise a receiving computer configured to receive temperature data and identity data from each container tracking device in the plurality of container tracking devices, to calculate permission codes based on the temperature data and the identity data, and to selectively transmit the permission codes to the container tracking devices in the plurality of container tracking devices.

Yet additional embodiments of the present invention relate to methods for tracking a container, one such method comprising: obtaining a system for tracking a plurality of containers such as those described herein; selecting a first container from the plurality of containers, the first container having coupled to it a first container tracking device from the plurality of container tracking devices; registering the first container tracking device to the identifying device for a donor to obtain an identity of the donor; receiving in the interior of the first container a fluid; measuring, using the first container tracking device, a first exterior temperature of the first container that correlates to a temperature of the fluid in the interior of the first container; transmitting from the first container tracking device to the receiving computer the identity of the donor and the first exterior temperature; periodically measuring, using the first container tracking device, subsequent exterior temperatures of the first container that correlate to subsequent temperatures of the fluid in the interior of the first container; timing a duration of the fluid in the first container, using the first container tracking device to obtain time data and transmitting the time data to the receiving computer, or using the receiving computer to obtain time data; periodically transmitting from the first container tracking device to the receiving computer the subsequent exterior temperatures; receiving at the receiving computer the identity of the donor, the first exterior temperature, and the subsequent exterior temperatures; recording at the receiving computer the identity of the donor, the first exterior temperature, the subsequent exterior temperatures, and a time elapsed since the receiving of the first temperature; optionally associating the identity of the donor with an identity of an intended recipient; causing the first container tracking device to interrogate an identity of a potential recipient when the first container tracking device and the potential recipient are brought in close proximity; transmitting the identity of the potential recipient from the first container tracking device to the receiving computer; receiving at the receiving computer the identity of the potential recipient; determining at the receiving computer whether the identity of the potential recipient matches the identity of the donor or optionally the identity of the intended recipient; comparing at the receiving computer the subsequent exterior temperatures over the time elapsed against an acceptable temperature profile; calculating a permission code based on the determining and the comparing; transmitting from the receiving computer to the first container tracking device the permission code; providing, at the first container tracking device, an alert based on the permission code.

Certain embodiments of the present invention relate to methods for managing a supply of fluid, one such method comprising: obtaining a system for tracking a plurality of containers such as those described; registering the plurality of containers to a donor to establish an identity of the donor; receiving in one or more containers in the plurality of containers, the fluid associated with the donor; transmitting from each of the one or more container tracking devices a signal when each containers receives the fluid; receiving at a receiving computer the signal from each of the one or more containers;

measuring a duration of the fluid in each container based on the receiving computer receiving the signal from each of the one or more container tracking devices; calculating a permission code for each container based on the measuring the duration of the fluid in each container; transmitting from the receiving computer the permission code for each container; providing, at each container tracking device for the one or more containers, an alert based on the permission code.

While the disclosure provides certain specific embodiments, the invention is not limited to those embodiments. A person of ordinary skill will appreciate from the description herein that modifications can be made to the described embodiments and therefore that the specification is broader in scope than the described embodiments. All examples are therefore non-limiting.

### BRIEF DESCRIPTION OF THE DRAWINGS

FIG. 1 is a block diagram conceptually illustrating the components of a container tracking device 101, according to one embodiment of the invention.

FIG. 2 illustrates a container tracking device 210 reversibly coupled to a bottle 201

FIG. 3 illustrates a container tracking device 310 permanently coupled to a bottle 301.

FIG. 4 illustrates a system 401 for ensuring proper handling and management of a plurality of containers according to one aspect of the present disclosure.

FIG. 5 depicts a flow diagram of a method for monitoring temperature and shelf life of a container, and confirming the identity of a user according to one aspect of the present disclosure.

FIG. 6 illustrates a container tracking device 610 coupled to a bottle 601 with one strap 670.

FIG. 7 illustrates a container tracking device 710 coupled to a bottle 701 with two straps 771, 772.

FIG. 8 illustrates another embodiment of a container tracking device 810 coupled to a bottle 801 with one strap 870.

FIG. 9 illustrates another embodiment of a container tracking device 910 coupled to a bottle 901 with two straps 971, 972.

### DETAILED DESCRIPTION

As required, detailed embodiments of the present invention are disclosed herein; however, it is to be understood that the disclosed embodiments are merely exemplary of the invention that may be embodied in various forms. The figures are not necessarily to scale, and some features may be exaggerated to show details of particular components. Therefore, specific structural and functional details disclosed herein are not to be interpreted as limiting, but merely as a basis for the claims and as a representative basis for teaching one skilled in the art to variously employ the present invention.

Unless defined otherwise, all technical and scientific terms used herein have the same meaning as is commonly understood by one of ordinary skill in the art to which this disclosure belongs. In the event that there is a plurality of definitions for a term herein, those in this section prevail unless stated otherwise.

Where ever the phrase “for example,” “such as,” “including” and the like are used herein, the phrase “and without limitation” is understood to follow unless explicitly stated otherwise. Similarly “an example,” “exemplary” and the like are understood to be non-limiting.

The term “substantially” allows for deviations from the descriptor that don't negatively impact the intended purpose. Descriptive terms are understood to be modified by the term “substantially” even if the word “substantially” is not explicitly recited.

The term “about” when used in connection with a numerical value refers to the actual given value, and to the approximation to such given value that would reasonably be inferred by one of ordinary skill in the art, including approximations due to the experimental and or measurement conditions for such given value.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a device having components a, b, and c” means that the device includes at least components a, b and c. Similarly, the phrase: “a method involving steps a, b, and c” means that the method includes at least steps a, b, and c.

Unless the context clearly requires otherwise, throughout the description and the claims, the words “comprise”, “comprising”, and the like are to be construed in an inclusive sense as opposed to an exclusive or exhaustive sense; that is to say, in the sense of “including, but not limited to”.

Any discussion of the prior art throughout the specification should in no way be considered as an admission that such prior art is widely known or forms part of common general knowledge in the field.

It is an object of the present invention to overcome or ameliorate at least one of the disadvantages of the prior art, or to provide a useful alternative.

One embodiment of the invention comprises a container tracking system developed to protect medical fluids from unsafe handling, improper storage, and misidentification. The medical fluids may comprise expressed breast milk, a prescription drug, a dietary supplement, a liquid diet food, an IV fluid, blood, plasma, tissue, a pharmaceutically-acceptable excipient, or a pharmaceutically-acceptable combination of two or more of the foregoing. For example, tissue such as cells useful for cancer treatment or gene therapy can be harvested from the donor, modified or enhanced for therapeutic utility, and administered to that donor or to a recipient. Similarly, tissue such as the blood or a component thereof from one donor can be harvested and administered to a recipient, as happened recently during the Ebola epidemic in Western Africa. Donors who had survived an Ebola infection donated blood in an effort to inoculate uninfected recipients; tracking that donated blood and its handling parameters such as age and temperature experience can be enhanced by employing one or more embodiments of the present invention.

According to another embodiment, the invention comprises a container tracking device comprising a temperature sensor configured to measure an exterior temperature of a container that correlates to a temperature of a fluid in the interior of the container. Any suitable temperature sensor can be used. In some cases, the temperature sensor is chosen from a thermocouple, a thermistor, a resistance temperature detector, an infrared sensor, and a combination thereof. Exterior temperature correlates to an interior temperature in any suitable manner. The temperature sensor can be calibrated, for example, by measuring a temperature of a fluid in the interior of the container using a thermometer, and observing the voltage, the current, or other property of the temperature positioned at the exterior of the container.

In another embodiment, the container tracking device comprises a proximity sensor configured to interrogate an identity of a donor or potential recipient when the container tracking device and the donor or potential recipient are brought in close proximity. Any suitable proximity sensor can be used. In some cases, the proximity sensor comprises a barcode reader, a radio-frequency identification reader, or both. As used herein, “close proximity” indicates any suitable distance. In certain instances, close proximity can mean within 0.5 feet, within one foot, within two feet, within five feet, within 10 feet, or within 20 feet. In other instances, close proximity can mean within the same room. Care should be taken so that the proximity sensor is not so sensitive as to trigger multiple negative alerts such as when a container is brought into a nursery. It is contemplated that when a medical professional seeks to administer a fluid to a recipient, the medical professional will first present the recipient's identifying device to the proximity sensor so the proximity sensor can interrogate the potential recipient's identifying device, in some cases. In other cases, the proximity sensor will interrogate the potential recipient's identifying device once the container is close enough to the potential recipient for administration. Interrogating the identity of a potential recipient can occur through any suitable protocol, such as, for example, the proximity sensor reads a barcode on the potential recipient's identifying device which is a wristband, for example. In another example, the proximity sensor detects the radio frequency identification chip associated with a potential recipient's identifying device.

In yet another embodiment, the container tracking device further comprises a timer communicatively coupled to the processor, which is configured to provide time data for monitoring the age of the container contents as well as providing a means to compare the dates of the containers, ensuring the oldest container is consumed first, in some cases. Alternatively, it may be desirable to ensure that the newest container is consumed first. In still another alternative, the timer can assist in identifying containers that have remained beyond their expiration date, and should not be administered.

In still another embodiment, a processor is communicatively coupled to the temperature sensor and the proximity sensor, wherein the processor is configured to receive temperature data from the temperature sensor and identity data from the proximity sensor. Any suitable processor can be used, such as, for example, those commercially available. Memory, which if present is communicatively coupled to the processor, is configured to store at least temperature data and identity data. In some cases, the container tracking device is configured to retain the identity of the donor. In further cases, the container tracking device is configured to retain a “timestamp” in the form of start time data corresponding to initially receiving the fluid to be stored in the container. In certain instances, memory coupled to the processor can store that data. Any suitable memory can be used. Solid-state non-volatile memory such as flash memory may be mentioned.

A transmitter is communicatively coupled to the processor, wherein the transmitter is configured to send temperature data and identity data to a receiving computer. Any suitable transmitter can be used. In some cases, the transmitter is chosen from short-wavelength UHF radio wave transmitters, wireless local area network transmitters, infrared transmitters, cellular network transmitters, and combinations thereof.

A receiver is communicatively coupled to the processor, wherein the receiver is configured to receive a permission code from the receiving computer. Any suitable receiver can be used. In some cases, the receiver is chosen from short-wavelength UHF radio wave receivers, wireless local area network receivers, infrared receivers, cellular network receiver, and combinations thereof.

An alert system is communicatively coupled to the processor, and is configured to provide an alert based on the permission code. Any suitable alert system can be used. In some cases, the alert system comprises at least one LED. When the permission code received is positive, a green LED is configured to illuminate, and when the permission code is negative, a red LED is configured to illuminate, in certain instances. Or, when only one LED is present, the one LED would illuminate only upon receipt of a positive permission code. In other instances, the alert system comprises a speaker, a bell, a vibrating alert such as a buzzer, or a combination thereof, configured to emit a first sound with the permission code is positive, and a second sound when the permission code is negative. The first sound and the second sound can be any suitable distinct sounds. For example, the first sound could be a single tone and the second sound can be pulses. In another example, the first sound could have a pleasant quality, and the second sound can have an unpleasant quality. Care should be taken, however, so that neither the first sound nor the second sound unduly disturb other persons, so that the entire nursery is not awakened, for example.

Various components of the container tracking device are “communicatively coupled.” That means that electronic signals representing data or instructions can be passed back and forth. For example, the temperature sensor provides temperature data to the processor, and then the processor instructs the transmitter to transmit that temperature data to the receiving computer. In that instance, the temperature sensor is communicatively coupled to the processor, and the processor is communicatively coupled to the transmitter.

According to another embodiment, the container tracking device is configured to be reversibly coupled to a container. In such cases, it may be beneficial to remove the container tracking device from the container for cleaning or sterilizing the container. Or, in other cases, the container tracking device is permanently coupled to the container. Care should be taken in such cases so that the container tracking device can endure all of the conditions of cooling, heating, storing, and cleaning the container. Any suitable means for coupling, reversibly or permanently, can be used, alone or in combination. Straps employing hook-and-loop fabrics such as Velcro®, snaps, buckles, and the like can be employed in some cases. Additionally, one or more rings or loops may be affixed to the strap or straps to serve as an attachment point for securing the strap or straps to the container.

In some cases, the location of the device on the container may be adjustable, for relocating the device to a desirable area of the container. In such cases, the device may be located at a lower portion of the container to monitor the temperature of the fluid during heating or cooling, or otherwise while the container is positioned so that the fluid is located in the lower portion of the container. Alternatively or in addition, the device can be relocated toward the top of the container to monitor the temperature of the fluid while dispensing and the container is tilted or inverted, placing the fluid near the top. A container tracking device that can be relocated to various locations on the container can be reversibly coupled to the container, or it can be permanently coupled to the container. In other cases, structure on the exterior of the container can be employed so the container tracking device “snaps” into place on the container. In still other cases, adhesive or tape can be used to affix the container tracking device to the container. In the example of expressed breast milk, the container the device may be attached to is a baby bottle, or a fluid storage bag, wherein the container tracking device is waterproof. Or, the device may be permanently coupled to a baby bottle, or a fluid storage bag, such that it is encased in a plastic covering within the bottle in certain instances.

Any suitable container can be used in the present invention. A container can be a baby bottle, a fluid storage bag, or the like, and they can be used in combination. For example, containers including baby bottles and fluid storage bags can be used for the collection, storage, and administration of expressed breast milk. The expressed breast milk can be received into fluid storage bags equipped with suitable container tracking devices and stored in a refrigerator. Then, when it is time to administer the breast milk to the infant, the milk can be transferred from the fluid storage bag to a baby bottle. The container tracking devices in that instance can assist with recording the duration of the milk in the fluid storage bag and the baby bottle, and ensuring the milk is administered to the correct infant. Any suitable fluid storage bag can be used. In some cases, a fluid storage bag is chosen from reclosable zip-top storage bags and intravenous fluid bags.

Fluid within a container of the present invention can be dispensed according to any suitable means. In some cases, the fluid can be simply poured out of the container. In other cases, the container is adapted to dispense the fluid in the interior of the container via a nipple or a cannula. Certain instances of the present invention provide fluid being received and stored in a first container, and administered a second container. For example, breast milk can be collected in a fluid storage bag such as, for example, a reclosable zip-top plastic bag, and stored in a refrigerator. Then, the milk can be transferred to a bottle to be given to the baby. In some cases, a container tracking device is reversibly coupled to the fluid storage bag and registered to the mother. When the milk is transferred to the bottle, the container tracking device can be removed from the fluid storage bag and reversibly coupled to the bottle. The container tracking device in this example can monitor the age and temperature of the milk during the entire time between expression and administration. Alternatively, a first container tracking device can record time data and temperature data while the fluid is in a first container, and then a second container tracking device can record time data and temperature data once the fluid is transferred to a second container. The receiving computer can assist with correlating the data from the first container tracking device with the data from the second container tracking device.

Certain embodiments of the present invention include a container tracking device having a power supply. Any suitable power supply can be used, alone or in combination. In some cases, a rechargeable or non-rechargeable lithium ion battery can be used. In other cases, a small solar cell can collect ambient light to recharge or power the device.

The invention further relates to a system for tracking a plurality of containers, wherein each container is adapted to receive, store, and dispense a fluid. One such system comprises a plurality of containers each comprising a container tracking device; an identifying device for a donor; optionally, an identifying device for an intended recipient; and a receiving computer. Any suitable containers, container tracking devices, and identifying devices for the donors and recipients can be used, such as those described herein. Any suitable receiving computer also can be used. The identifying devices for the donor and optionally the intended recipient can be alike or different, and can comprise a barcode, a radio frequency identification device, or both. The identifying devices for the donor and recipient can be configured to be semi-permanently attached to the donor and recipient, respectively. “Semi-permanently attached” indicates the identifying device cannot be easily removed from the donor or recipient, but does not unduly discomfort the donor or recipient. For example, the identifying device such as a barcode or a radio frequency identification device can be affixed to a wristband or an ankle band to be worn by the donor or recipient. Conventional plastic wristbands and ankle bands cannot be removed from the donor or recipient without cutting or damaging the band, and so are semi-permanently attached to the donor or recipient once deployed.

Optionally, the system also comprises an identifying device for an intended recipient, and the identifying device for the intended recipient is configured to be interrogated by the proximity sensor of each container tracking device in the plurality of container tracking devices. Optionally, the donor is related to the intended recipient, as in the example of a mother and her infant, or a tissue donor and the tissue recipient. In the case of a tissue donor and a tissue recipient, they can be pre-matched or not. A donor and recipient can be pre-matched by any suitable means, such as blood type testing or other conventional organ-donor matching protocols. Or, the donor can be supplying milk, blood, or other tissue for one or more recipients to be identified later. In such cases, the milk, blood, or other tissue can be banked for future use. In the example of expressed breast milk, the containers may comprise baby bottles wherein the fluid comprises a substance received from the donor, the mother, to be administered to the recipient, the infant. The identifying device for the donor comprises a barcode, a radio frequency identification device, or both. The identifying device for the donor is configured to be semi-permanently attached to the donor, wherein the identifying device is affixed to a wristband or an ankle band to be worn by the donor. The identifying device for the recipient comprises a barcode, a radio frequency identification device, or both. The identifying device for the recipient is configured to be semi-permanently attached to the recipient, wherein the identifying device is affixed to a wristband or an ankle band to be worn by the recipient.

Additionally, another embodiment relates to a system comprising an identifying device for a recipient, where the recipient is not the donor's infant. In the example of the expressed breast milk, the containers may comprise baby bottles wherein the fluid comprises a substance received from the donor to be administered to the recipient. The recipient may be a particular infant designated to receive that expressed breast milk. That donor and that recipient have been pre-matched. Alternatively, the infant could be administered expressed breast milk in a different context employing the system such as from a milk bank.

Any suitable receiving computer can be used in the various embodiments of the present invention. A receiving computer is configured to receive temperature data, identity data, and optionally time data from each container tracking device in the plurality of container tracking devices to calculate permission codes based on the temperature data, the identity data, and optionally the time data, and to selectively transmit the permission codes to the container tracking devices in the plurality of container tracking devices. In some alternatives, the receiving computer itself measures the time data. Furthermore, each container tracking device is further configured to selectively receive the permission codes, and provide alerts based on the permission codes.

In a further aspect, the receiving computer is configured to communicate with the plurality of container tracking devices. Communication between the receiving computer and the container tracking devices can occur according to any suitable technology. For example, a receiving computer and container tracking devices can transmit and receive information via short-wavelength UHF radio waves such as Bluetooth®, a wireless local area network such as Wi-Fi, infrared, a cellular network, or a combination thereof. In certain instances, the receiving computer is chosen from a server, a desktop computer, a tablet computer, a wearable computer such as Google Glass® and i-Watch®, and a smart phone. In other instances, the functions of the receiving computer can be distributed over a network of such devices.

According to another embodiment of the present disclosure, a method for tracking a container comprises obtaining a system for tracking a plurality of containers such as any of those systems described herein. Then, a first container is selected from the plurality of containers, the first container having coupled to it a first container tracking device from the plurality of container tracking devices. The first container tracking device is registered to the identifying device for a donor to obtain an identity of the donor. Registering the first container tracking device to the identifying device for the donor can include any suitable protocol such as, for example, scanning the identifying device for the donor to obtain the identity of the donor; and transmitting from the first container tracking device to the receiving computer the identity of the donor.

A fluid is received in the interior of the first container and a first exterior temperature of the first container is measured, using a temperature sensor on the first container tracking device. A signal indicating the time that the container receives the fluid is transmitted from the container tracking device to the receiving computer, in some cases; in other cases, the receiving computer notes the time at which the first exterior temperature is received at the receiving computer. The signal indicating a container is receiving a fluid can be triggered by any suitable means. In some cases, the container tracking device will comprise a button or other manual switch allowing a medical professional to indicate the container is now receiving fluid. In other cases, a change in temperature can be used to note that the container is now receiving fluid. The identity of the donor and the first exterior temperature is transmitted from the first container tracking device to the receiving computer. In addition, the container tracking device will periodically measure subsequent exterior temperatures of the first container that correlate to subsequent temperatures of the fluid in the interior of the first container. The subsequent exterior temperatures are periodically transmitted from the first container tracking device to the receiving computer. Accordingly, the receiving computer receives the identity of the donor, the first exterior temperature, optionally a signal indicating the container is receiving fluid, and the subsequent exterior temperatures. The identity of the donor, the first exterior temperature, the subsequent exterior temperatures, and a time elapsed since the receiving of the first temperature are recorded at the receiving computer. Optionally, the receiving computer may associate the identity of the donor with an identity of an intended recipient. In some cases, the container tracking device retains the identity of the donor, a start time data indicating when fluid was first received in the corresponding container, or both.

When it is time to administer the fluid in the first container, the first container with its first container tracking device is brought in close proximity with a potential recipient. The first container tracking device interrogates an identity of a potential recipient's identifying device. The first container tracking device transmits the identity of the potential recipient to the receiving computer. The receiving computer receives the identity of the potential recipient and determines whether the identity of the potential recipient matches the identity of the donor or optionally the identity of the intended recipient.

The receiving computer compares the subsequent exterior temperatures over the time elapsed against an acceptable temperature profile by correlating the subsequent exterior temperatures with the subsequent temperatures of the fluid in the interior of the first container; and comparing the subsequent temperatures of the fluid in the interior of the first container against the acceptable temperature profile. Any suitable temperature profile can be established. In some cases, an acceptable temperature profile looks only for temperature extremes. In other cases, an acceptable temperature profile correlates the time a fluid has a temperature within particular ranges to determine whether acceptable temperatures have been exceeded. For example, expressed breast milk can be stored at room temperature for an hour or so, or at refrigerator temperatures for several days, or in a frozen state for several months. A sophisticated acceptable temperature profile can be developed so that milk having a complicated temperature experience can still be identified as acceptable or unacceptable. Still further cases allow for an evaluation of a final temperature just before administration. For example, breast milk taken from a freezer or a refrigerator should be heated to room temperature or above, but not above about 100° F. An acceptable temperature profile can also evaluate whether the milk experienced so great a temperature in the heating that the milk is no longer acceptable, in certain instances. Thus, the receiving computer can determine whether the fluid has been heated above an acceptable heating temperature when calculating a permission code. In further instances, an acceptable temperature profile can evaluate whether the milk is currently at an acceptable administration temperature for an infant to consume. Similarly, IV fluids have acceptable storage temperature experiences and acceptable administration temperatures that can be used to establish acceptable temperature profiles. Therefore, a permission code may be calculated by searching for subsequent temperatures outside of a predetermined temperature range to identify one or more temperature extremes, and determining whether at least one of the one or more temperature extremes exceeds an acceptable duration, in some embodiments. The receiving computer transmits to the first container tracking device the permission code. The container tracking device then provides an alert based on the permission code.

The receiving computer measures a duration of the fluid in each container, in certain cases based on a signal received from each container tracking device. A permission code is calculated by comparing the durations. In some cases, the receiving computer can determine whether at least one of the one or more durations exceeds an acceptable duration, thereby indicating the fluid in that container has “expired” and should not be administered. The receiving computer transmits to the first container tracking device the permission code, providing, at the first container tracking device, an alert based on the permission code.

In one embodiment, the permission code for each container is based on the measuring the duration of the fluid in each container and comprises calculating a positive permission code for the container having the longest duration. This would allow for the first container, or the container with the longest duration, to be identified and used prior to using fresher containers. In another embodiment, the permission code for each container is based on the measuring the duration of the fluid in each container and comprises calculating a positive permission code for the container having the shortest duration. This would allow for the last container, or the container with the shortest duration, to be identified and used prior to using older containers.

In yet another embodiment, the permission code for each container is based on the measuring the duration of the fluid in each container and comprises calculating a negative permission code for each container having a duration exceeding a predetermined duration. The negative permission code causes the providing, at each container tracking device for the one or more containers, a permanent negative alert based on the negative permission code for each container having the duration exceeding the predetermined duration. This would allow for the container that has exceeded the time duration to be identified as expired.

### DETAILED DESCRIPTION OF THE DRAWINGS

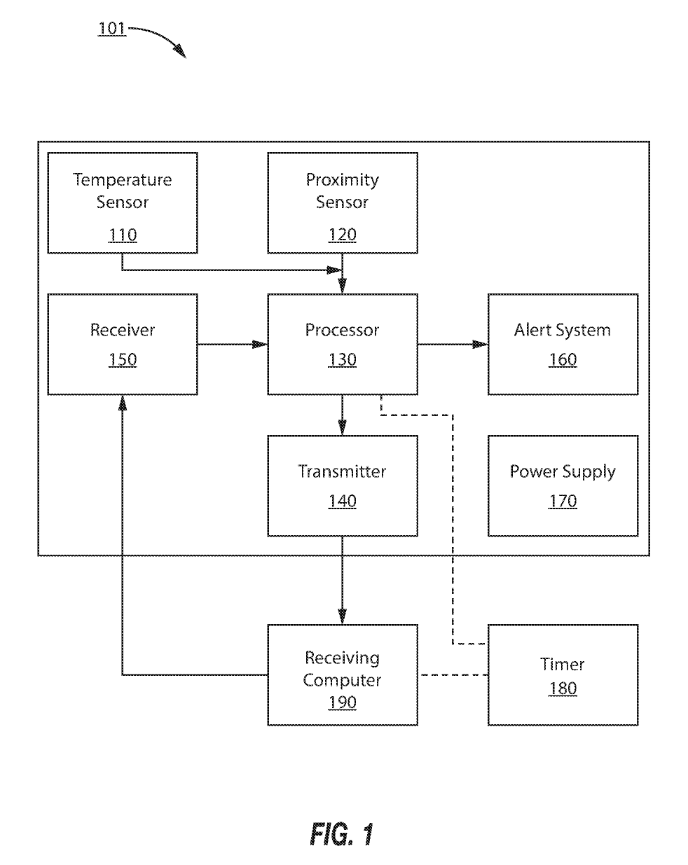

FIG. 1 provides a schematic of an embodiment of the present invention, container tracking device 101. In this figure, a container tracking device 101 includes a temperature sensor 110 for detecting the exterior temperature of a container that correlates to a temperature of fluid in the interior of the container. Additionally, container tracking device 101 includes proximity sensor 120 for interrogating an identity of a donor or a potential recipient when the container tracking device and the donor or the potential recipient are brought in close proximity. Temperature sensor 110 and proximity sensor 120 are communicatively coupled to processor 130 which is configured to receive temperature data from temperature sensor 110 and identity data from proximity sensor 120. Transmitter 140 is communicatively coupled to processor 130, and is configured to send the data to receiving computer 190. Receiving computer 190 is configured to receive that data from the container tracking device 101, calculate a permission code based on the temperature data and the identity data, and send the permission code to receiver 150, which is coupled to processor 130. An alert system 160 is coupled to processor 140 and is configured to provide an alert based on the permission code. Container tracking device 101 is powered by power supply 170.

Optional timer 180 can be part of container tracking device 101 or receiving computer 190. In either case, timer 180 can represent separate structure or can be performed by a suitable process, such as, for example, a process encoded on software to be performed by processor 130 or on receiving computer 190. Timer 180 can be communicatively coupled to the processor 130. Whether part of container tracking device 101 or receiving computer 190, timer 180 is configured to provide time data such as a start time data for monitoring the age of the contents of a container (not shown). Also, timer 180 can assist with comparing the dates of different containers (not shown), ensuring the oldest container is consumed first, in some cases.

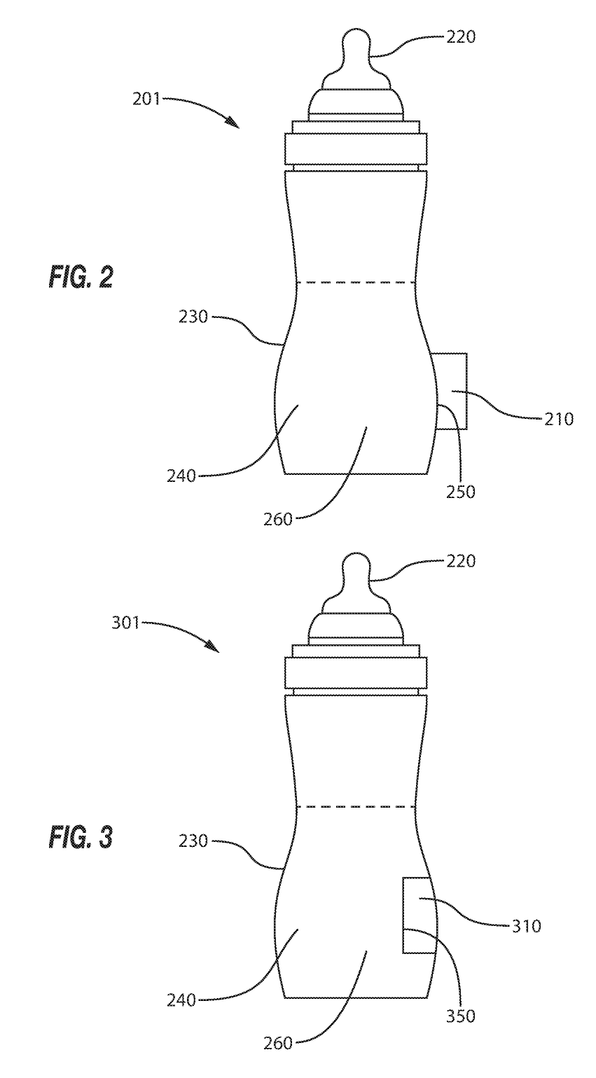

FIG. 2 schematically represents container tracking device 210 reversibly coupled to container 201. Container 201 comprises a nipple 220 for dispensing fluid 260 from the interior 240. Also shown is exterior 230 of container 201. At surface 250, a temperature sensor (not shown) of container tracking device 210 is configured to measure an exterior temperature of container 201 that correlates to a temperature of fluid 260 in the interior 240 of container 201. Additionally, a proximity sensor (not shown) of container tracking device 210 is configured to interrogate an identity of a donor (not shown) or a potential recipient (not shown) when the container tracking device 210 and the donor or potential recipient are brought in close proximity. Container tracking device 210 is configured to be reversibly coupled to container 201, so that container can be cleaned or sterilized without damaging container tracking device 210. Optionally, a timer (not shown) of container tracking device 210 is configured to provide time data for monitoring the age of the fluid 260 in interior 240 of container 201.

FIG. 3 schematically represents container tracking device 310 permanently coupled to container 301. Similar to container 201, container 301 comprises a nipple 220 for dispensing fluid 260 from the interior 240. Also shown is surface 350 where a temperature sensor (not shown) of container tracking device 310 is configured to measure an exterior temperature of container 301 that correlates to a temperature of fluid 260 in the interior 240 of container 301. Additionally, a proximity sensor (not shown) of container tracking device 310 is configured to interrogate an identity of a donor (not shown) when the container tracking device 310 and the donor are brought in close proximity. Container tracking device 310 is configured to be permanently coupled to container 301. Optionally, a timer (not shown) of container tracking device 310 is configured to provide time data for monitoring the age of the fluid 260 in interior 240 of container 301.

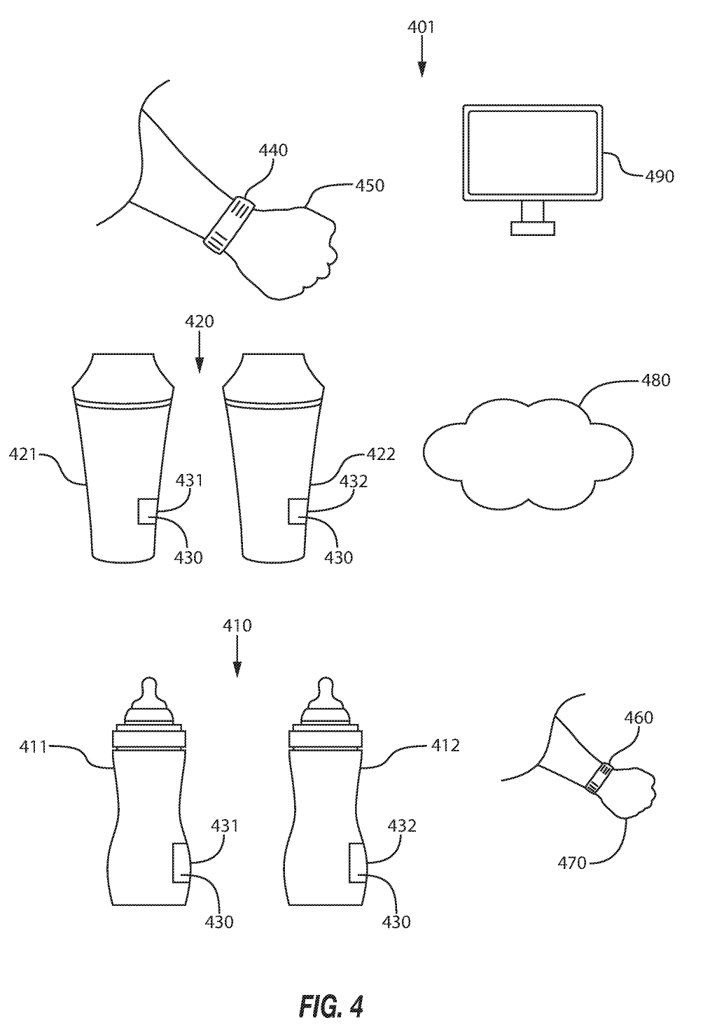

FIG. 4 illustrates a system 401 for tracking a plurality 410, 420 of containers 411, 412, 421, 422. System 401 is shown to include a plurality 420 of bags 421, 422 and a plurality 410 of bottles 411, 412, which are adapted to receive, store, and dispense a fluid (not shown). Initially, fluid storage bag 421 comprises container tracking device 431 reversibly coupled to fluid storage bag 421. Container tracking device 432 is reversibly coupled to fluid storage bag 422. A fluid, such as, for example, expressed breast milk, is transferred from donor 450 to fluid storage bag 421 selected from the plurality 420, with container tracking device 431 attached. The container tracking device 431 is registered to donor 450 by any suitable protocol, such as having the proximity sensor (not shown) of container tracking device 431 interrogate identifying device 440 for donor 450. Alternatively, the plurality 410, 420 of containers can be registered to donor 450 at the time the identifying device 440 is semi-permanently attached to the wrist of donor 450. Once the milk enters fluid storage bag 421, container tracking device 431 is configured to measure an exterior temperature of fluid storage bag 421 that correlates to a temperature of fluid in the interior of fluid storage bag 421. Container tracking device 431 is optionally configured with a timer (not shown) to time a duration of fluid in fluid storage bag 421 to obtain time data. A signal indicating the initial temperature and the time that fluid storage bag 421 receives the fluid is transmitted to receiving computer 490 through network 480. Network 480 can be any suitable network, such as, for example, short-wavelength UHF radio wave network such as Bluetooth®, a wireless local area network such as Wi-Fi, infrared network, a cellular network, or a combination thereof. Receiving computer 490 is configured to receive temperature data, identity data, and optionally time data from container tracking device 431 to calculate permission codes for fluid storage bag 421 based on the temperature data, the identity data, and optionally the time data, and to selectively transmit the permission codes to container tracking device 431. Furthermore, container tracking device 431 is further configured to selectively receive the permission codes, and provide alerts based on the permission codes. Thus, if fluid storage bag 421 experiences an extreme temperature, or endures an otherwise acceptable temperature for too long a duration, container tracking device 431 could receive a negative permission code from receiving computer 490 via network 480, and provide a negative alert such as a red light or an unpleasant sound.

In this embodiment, the fluid is transferred from fluid storage bag 421 and poured into bottle 411 selected from the plurality of bottles 410. Container tracking device 431 also is removed from fluid storage bag 421 and is reversibly coupled to bottle 411. Now, container tracking device 431 is configured to measure an exterior temperature of bottle 411 that correlates to a temperature of fluid in the interior of bottle 411. Container tracking device 431 is optionally configured to with a timer (not shown) to time a duration of fluid in bottle 411 to obtain time data. A signal indicating the initial temperature and the time that bottle 411 receives the fluid is transmitted to receiving computer 490 through network 480. Also, subsequent temperature data is collected by container tracking device 431 and transmitted to receiving computer 490. Receiving computer 490 is configured to receive temperature data, any identity data, and optionally time data from container tracking device 431 in the plurality of container tracking devices 430 to calculate permission codes based on the temperature data, the identity data, and optionally the time data, and to selectively transmit the permission codes to container tracking device 431 in the plurality of container tracking devices 430. Furthermore, container tracking device 431 is further configured to selectively receive the permission codes, and provide alerts based on the permission codes.

Prior to administering the fluid in container 411 to intended recipient 470, identifying device 460 is configured to be interrogated by the proximity sensor (not shown) of container tracking device 431 to confirm identity. Receiving computer 490 is configured to receive temperature data, identity data and optionally time data from container tracking device 431 to calculate permission codes. Time data can be recorded internally in receiving computer 490, if container tracking device 431 does not include a timer. Receiving computer 490 transmits the permission codes to container tracking device 431, which provides an alert based on the permission codes.

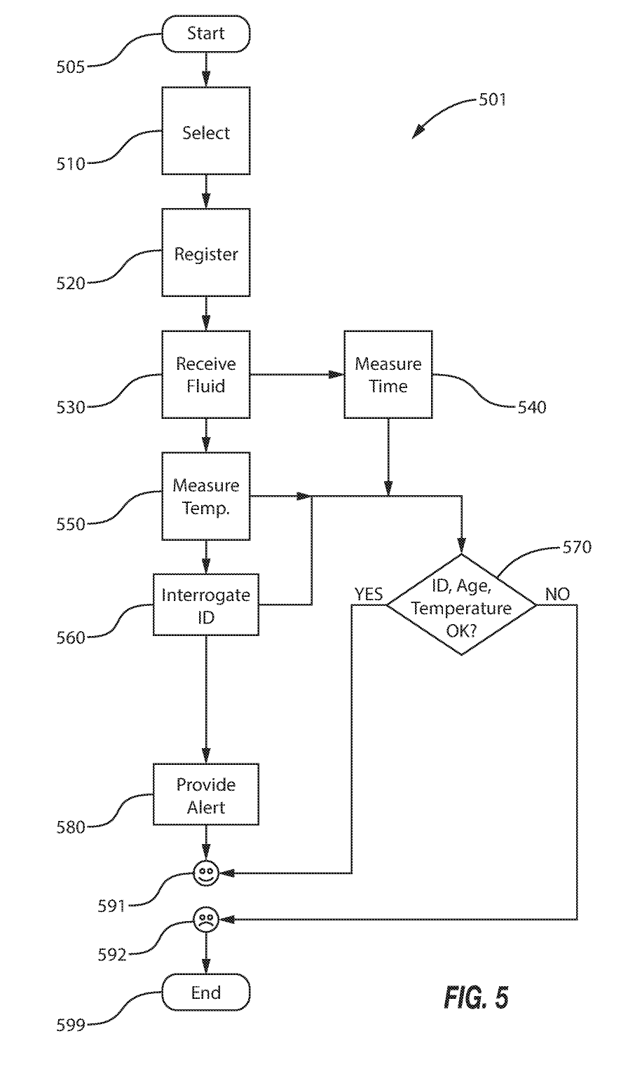

FIG. 5 is a flowchart illustrating method 501 for tracking a container. To start 505, a container with a container tracking device coupled to it is selected 510 from the plurality of containers in FIG. 4. Then, a container tracking device is registered 520 to a donor for example by interrogating the identifying device for the donor. The container then receives any suitable fluid comprising a substance from the donor at step 530. The initial time the container receives fluid is measured at step 540 and transmitted to the receiving computer, or is otherwise recorded at the receiving computer. The initial exterior temperature of the container is measured at step 550 and transmitted to the receiving computer. Subsequent exterior temperatures are periodically measured at step 550 using the container tracking device and are transmitted to the receiving computer. The duration that the fluid is in the container is measured at step 540 using the container tracking device and transmitted to the receiving computer, or is otherwise recorded at the receiving computer. Steps 540 and 550 are independently periodic, and continue for so long as fluid is stored in the container, until a permanent negative permission code is transmitted and received for the container tracking device, or until the container tracking device is reset according to any suitable protocol. At step 560, the container tracking device interrogates the identity of a potential recipient when the container is brought into close proximity to the potential recipient prior to administering the fluid in the container. The container tracking device transmits the identity of the potential recipient to the receiving computer, and the receiving computer determines whether the identity of the potential recipient matches that of the intended recipient. At step 570, the receiving computer compares the subsequent exterior temperatures over the time elapsed against the acceptable profile, compares the identity of the potential recipient with the identity of the intended recipient, and calculates a positive permission code or a negative permission code. At step 580, the container tracking device provides an alert based on the permission code. The alert could be a positive alert 591 which may be a green light or a first more pleasant sound. Alternatively, the alert could be a negative alert 592 which may be a red light or a second less pleasant sound. At the end 599 the fluid is administered to the proper, confirmed, intended recipient.

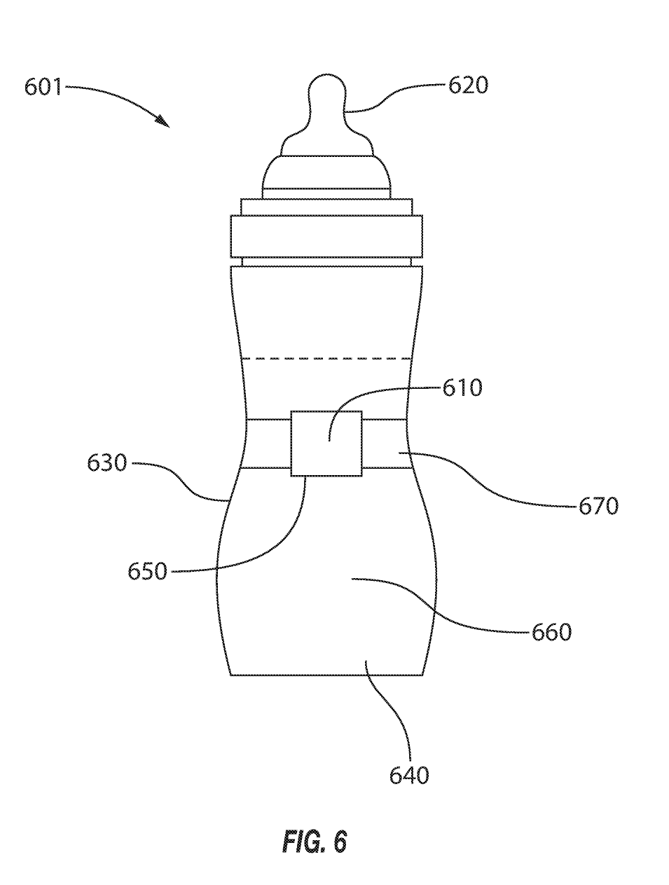

FIG. 6 schematically represents container tracking device 610 coupled to container 601. Container 601 comprises a nipple 620 for dispensing fluid 660 from the interior 640. Also shown is exterior 630 of container 601. At surface 650, a temperature sensor (not shown) of container tracking device 610 is configured to measure an exterior temperature of container 601 that correlates to a temperature of fluid 660 in the interior 640 of container 601. Additionally, a proximity sensor (not shown) of container tracking device 610 is configured to interrogate an identity of a donor (not shown) or a potential recipient (not shown) when the container tracking device 610 and the donor or potential recipient are brought in close proximity. Container tracking device 610 can be permanently coupled to container 601, or it can be configured to be reversibly coupled to container 601, with one strap 670 affixed in a horizontal position on the exterior 630 of container 601, so that container can be cleaned or sterilized without damaging container tracking device 610. Strap 670 can extend around the entire circumference of container 601 and attach to itself and/or container 601 with, for example, one or more rings, loops, hook-and-loop fabric, snaps, buckles, or combinations thereof. In addition or alternatively, structures forming anchor points for strap 670 can be built into container 601. In some cases, container tracking device 610 and strap 670 are configured to allow device 610 to be moved over the exterior 630 of container 601. In this way, the location of the container tracking device 610 on the container 601 is adjustable. The container tracking device 610 therefore can be moved to reflect the level of fluid 660 in container 601. Optionally, a timer (not shown) of container tracking device 610 is configured to provide time data for monitoring the age of the fluid 660 in interior 640 of container 601.

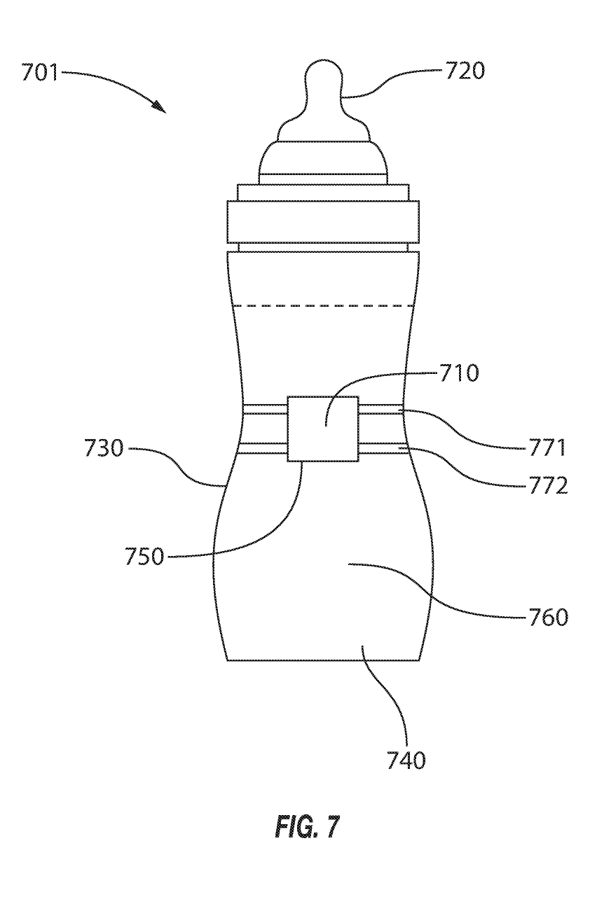

FIG. 7 schematically represents container tracking device 710 coupled to container 701. Container 701 comprises a nipple 720 for dispensing fluid 760 from the interior 740. Also shown is exterior 730 of container 701. At surface 750, a temperature sensor (not shown) of container tracking device 710 is configured to measure an exterior temperature of container 701 that correlates to a temperature of fluid 760 in the interior 740 of container 701. Additionally, a proximity sensor (not shown) of container tracking device 710 is configured to interrogate an identity of a donor (not shown) or a potential recipient (not shown) when the container tracking device 710 and the donor or potential recipient are brought in close proximity. Container tracking device 710 can be permanently coupled to container 701. Or, container tracking device 710 is configured to be reversibly coupled to container 701 with two straps 771, 772 affixed in a horizontal position on the exterior 730 of container 701, so that container can be cleaned or sterilized without damaging container tracking device 710. Optionally, a timer (not shown) of container tracking device 710 is configured to provide time data for monitoring the age of the fluid 760 in interior 740 of container 701. Straps 771, 772 can employ at least one attachment means to affix the two straps 771, 772 to container 701. That attachment means, chosen for example from rings, loops, hook-and-loop fabric, snaps, buckles, and combinations thereof, can allow the location of the container tracking device 710 on the container 701 to be adjustable, in some cases.

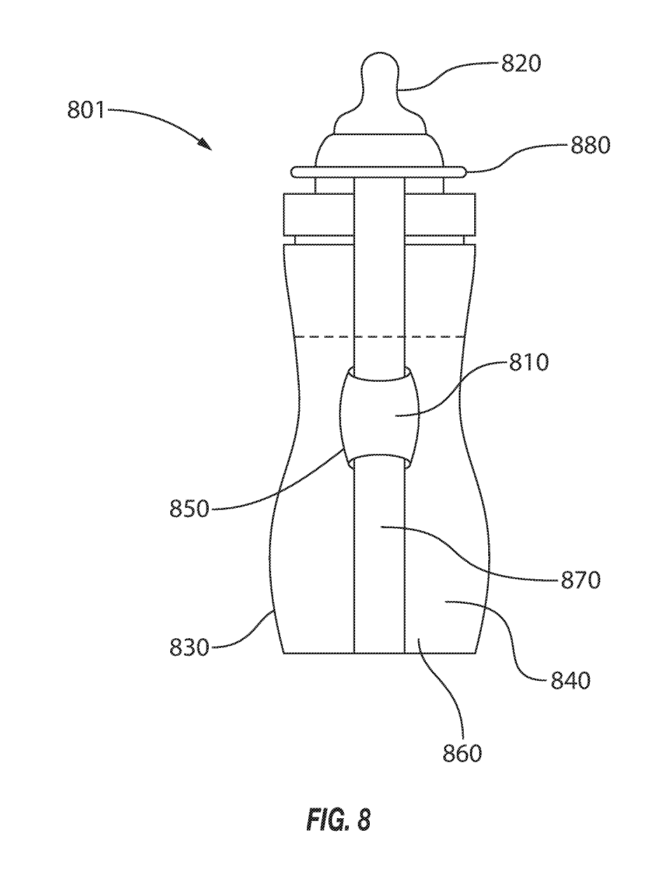

FIG. 8 schematically represents container tracking device 810 coupled to container 801. Container 801 comprises a nipple 820 for dispensing fluid 860 from the interior 840. Also shown is exterior 830 of container 801. At surface 850, a temperature sensor (not shown) of container tracking device 810 is configured to measure an exterior temperature of container 801 that correlates to a temperature of fluid 860 in the interior 840 of container 801. Additionally, a proximity sensor (not shown) of container tracking device 810 is configured to interrogate an identity of a donor (not shown) or a potential recipient (not shown) when the container tracking device 810 and the donor or potential recipient are brought in close proximity. Container tracking device 810 can be permanently affixed to container 801. Or, container tracking device 810 can be configured to be reversibly coupled to container 801 with one strap 870 attached to container 801 affixed in a vertical position to the exterior 830 of container 801, with securing ring 880 placed over nipple 820, so that container can be cleaned or sterilized without damaging container tracking device 810. Strap 870 allows the location of container tracking device 810 on container 801 to be adjustable, so that the donor or a health care worker can move the container tracking device 810 up and down the container 801 along strap 870 to where the fluid 860 is in the interior 840. That can allow more-accurate temperature readings when, for example, the container 801 is tilted or inverted and the fluid is near nipple 820. Accordingly, certain embodiments of the present invention allow temperature monitoring while fluid 860 is being dispensed. Optionally, a timer (not shown) of container tracking device 810 is configured to provide time data for monitoring the age of the fluid 860 in interior 840 of container 801.

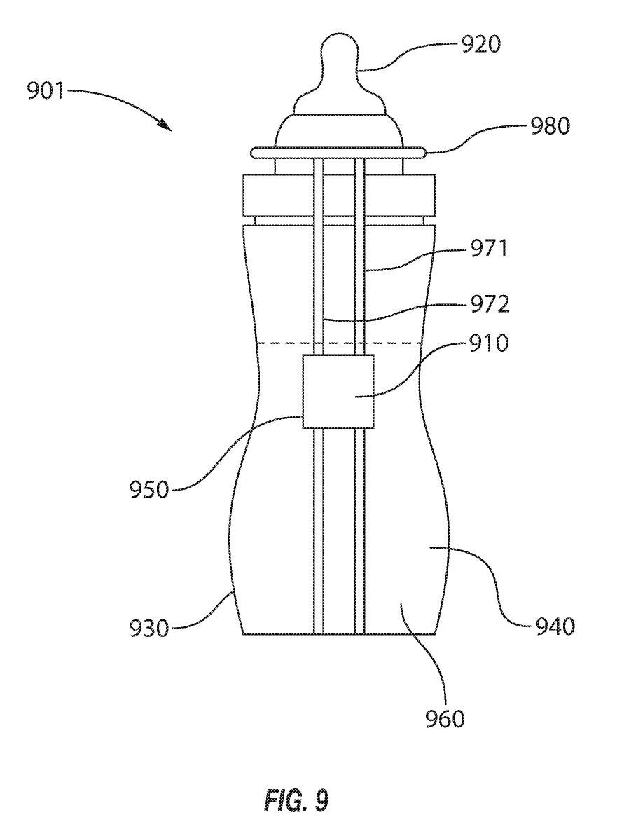

FIG. 9 schematically represents container tracking device 910 coupled to container 901. Container 901 comprises a nipple 920 for dispensing fluid 960 from the interior 940. Also shown is exterior 930 of container 901. At surface 950, a temperature sensor (not shown) of container tracking device 910 is configured to measure an exterior temperature of container 901 that correlates to a temperature of fluid 960 in the interior 940 of container 901. Additionally, a proximity sensor (not shown) of container tracking device 910 is configured to interrogate an identity of a donor (not shown) or a potential recipient (not shown) when the container tracking device 910 and the donor or potential recipient are brought in close proximity. Container tracking device 910 can be permanently coupled to device 901. Or, container tracking device 910 is configured to be reversibly coupled to container 901 with two straps 971, 972 attached to container 901 and affixed in a vertical position on the exterior 930 of container 901, with securing ring 980 placed over nipple 920, so that container can be cleaned or sterilized without damaging container tracking device 910. Straps 971, 972 allow the location of container tracking device 910 on container 901 to be adjustable, so that the donor or a health care worker can move the container tracking device 910 up and down the container 901 along straps 971, 972 to where the fluid 960 is in the interior 940. That can allow more-accurate temperature readings when, for example, the container 901 is tilted or inverted and the fluid is near nipple 920. Accordingly, certain embodiments of the present invention allow temperature monitoring while fluid 960 is being dispensed. Optionally, a timer (not shown) of container tracking device 910 is configured to provide time data for monitoring the age of the fluid 960 in interior 940 of container 901.

### Embodiments

Embodiment 1. A container tracking device, comprising:
a temperature sensor configured to measure an exterior temperature of a container that correlates to a temperature of a fluid in the interior of the container;
a proximity sensor configured to interrogate an identity of a donor or potential recipient when the container tracking device and the donor or potential recipient are brought in close proximity;
a processor which is communicatively coupled to the temperature sensor and the proximity sensor, the processor being configured to receive temperature data from the temperature sensor and identity data from the proximity sensor;
a transmitter communicatively coupled to the processor, the transmitter configured to send temperature data and identity data to a receiving computer;
a receiver communicatively coupled to the processor, the receiver configured to receive a permission code from the receiving computer;
an alert system communicatively coupled to the processor, the alert system configured to provide an alert based on the permission code; and
a power supply.
Embodiment 2. The container tracking device of embodiment 1, wherein the device is configured to be reversibly coupled to the container.
Embodiment 3. The container tracking device of embodiment 1, wherein the device is permanently coupled to the container.
Embodiment 4. The container tracking device of any one of embodiments 2 and 3, wherein the device is coupled to the container with a single strap affixed in a horizontal position on the exterior of the container.
Embodiment 5. The container tracking device of any one of embodiments 2 and 3, where in the device is coupled to the container with at least two straps affixed in a horizontal position on the exterior of the container.
Embodiment 6. The container tracking device of any one of embodiments 2 and 3, where in the device is coupled to the container with a single strap affixed in a vertical position on the exterior of the container.
Embodiment 7. The container tracking device of any one of embodiments 2 and 3, where in the device is coupled to the container with at least two straps affixed in a vertical position on the exterior of the container.
Embodiment 8. The container tracking device of any one of embodiments 4 and 6, wherein the single strap has at least one attachment means to affix the single strap to the container.
Embodiment 9. The container tracking device of any one of embodiments 5 and 7, wherein the at least two straps have at least one attachment means to affix the at least two straps to the container.
Embodiment 10. The container tracking device of any one of embodiments 8 and 9, wherein the at least one attachment means is chosen from rings, loops, hook-and-loop fabric, snaps, buckles, and combinations thereof.
Embodiment 11. The container tracking device of any one of embodiments 4-10, wherein the location of the container tracking device on the container is adjustable.
Embodiment 12. The container tracking device of any one of embodiments 1-11, wherein the temperature sensor is chosen from a thermocouple, a thermistor, a resistance temperature detector, an infrared sensor, and a combination thereof.
Embodiment 13. The container tracking device of any one of embodiments 1-12, wherein the fluid in the interior of the container comprises expressed breast milk, a prescription drug, a dietary supplement, a liquid diet food, an IV fluid, blood, plasma, tissue, a pharmaceutically-acceptable excipient, or a pharmaceutically-acceptable combination of two or more of the foregoing.
Embodiment 14. The container tracking device of any one of embodiments 1-13, wherein the container is adapted to dispense the fluid in the interior of the container via a nipple or a cannula.
Embodiment 15. The container tracking device of any one of embodiments 1-14, wherein the container is a baby bottle or a fluid storage bag.
Embodiment 16. The container tracking device of any one of embodiments 1-15, wherein the container is a fluid storage bag chosen from reclosable zip-top storage bags and intravenous fluid bags.
Embodiment 17. The container tracking device of any one of embodiments 1-16, wherein the proximity sensor comprises a barcode reader, a radio-frequency identification reader, or both.
Embodiment 18. The container tracking device of any one of embodiments 1-17, wherein the alert system comprises at least one LED.
Embodiment 19. The container tracking device of embodiment 18, wherein the alert system comprises a green LED configured to light when the permission code is positive, and a red LED configured to light when the permission code is negative.
Embodiment 20. The container tracking device of any one of embodiments 1-19, wherein the alert system comprises a speaker, a vibrating alert, a bell, or a combination thereof, configured to emit a first sound when the permission code is positive, and a second sound when the permission code is negative.
Embodiment 21. The container tracking device of any one of embodiments 1-20, further comprising a timer communicatively coupled to the processor, the timer configured to provide time data to the processor, and the transmitter is further configured to transmit the time data to the receiving computer.
Embodiment 22. The container tracking device of any one of embodiments 1-21, further comprising memory communicatively coupled to the processor, the memory configured to store at least temperature data and identity data.
Embodiment 23. The container tracking device of any one of embodiments 1-22, wherein the transmitter is chosen from short-wavelength UHF radio wave transmitters, wireless local area network transmitters, infrared transmitters, cellular network transmitters, and combinations thereof.
Embodiment 24. The container tracking device of any one of embodiments 1-23, wherein the receiver is chosen from short-wavelength UHF radio wave receivers, wireless local area network receivers, infrared receivers, cellular network receivers, and combinations thereof.
Embodiment 25. The container tracking device of any one of embodiments 1-24, wherein the container tracking device is waterproof.
Embodiment 26. The container tracking device of any one of embodiments 1-25, configured to retain identity data of a donor and a start time data.
Embodiment 27. A system for tracking a plurality of containers, comprising:
the plurality of containers, wherein each container is adapted to receive, store, and dispense a fluid;
a plurality of container tracking devices, each container tracking device in the plurality of container tracking devices being coupled to a container in the plurality of containers, each container tracking device being configured to measure an exterior temperature of the container that correlates to a temperature of a fluid in the interior of the container to obtain temperature data, and a proximity sensor configured to interrogate an identity of a donor or potential recipient when the container tracking device and the donor or potential recipient are brought in close proximity to obtain identity data;
an identifying device for a donor configured to be interrogated by the proximity sensor of each container tracking device in the plurality of container tracking devices;
optionally, an identifying device for an intended recipient, the identifying device for the intended recipient being configured to be interrogated by the proximity sensor of each container tracking device in the plurality of container tracking devices;
a receiving computer configured
to receive the temperature data and the identity data from each container tracking device in the plurality of container tracking devices,
to time a duration of the fluid in each container in the plurality of containers, based on the temperature data and the identity data received, to obtain time data,
to calculate permission codes based on the temperature data, the identity data, and the time data, and
to selectively transmit the permission codes to the container tracking devices in the plurality of container tracking devices,
wherein each container tracking device is further configured to selectively receive the permission codes, and provide alerts based on the permission codes.
Embodiment 28. A system for tracking a plurality of containers, comprising:
the plurality of containers, wherein each container is adapted to receive, store, and dispense a fluid;
a plurality of container tracking devices, each container tracking device in the plurality of container tracking devices being coupled to a container in the plurality of containers, each container tracking device being configured to measure an exterior temperature of the container that correlates to a temperature of a fluid in the interior of the container to obtain temperature data, a proximity sensor configured to interrogate an identity of a donor or potential recipient when the container tracking device and the donor or potential recipient are brought in close proximity to obtain identity data, and a timer configured to time a duration of the fluid in the interior of the container to obtain time data;
an identifying device for a donor configured to be interrogated by the proximity sensor of each container tracking device in the plurality of container tracking devices;
optionally, an identifying device for an intended recipient, the identifying device for the intended recipient being configured to be interrogated by the proximity sensor of each container tracking device in the plurality of container tracking devices;
a receiving computer configured
to receive the temperature data, the identity data, and the time data from each container tracking device in the plurality of container tracking devices,
to calculate permission codes based on the temperature data, the identity data, and the time data, and
to selectively transmit the permission codes to the container tracking devices in the plurality of container tracking devices,
wherein each container tracking device is further configured to selectively receive the permission codes, and provide alerts based on the permission codes.
Embodiment 29. The system of any one of embodiments 27-28, wherein each container in the plurality of containers is adapted to receive, store, and dispense a fluid comprising expressed breast milk, a prescription drug, a dietary supplement, a liquid diet food, an IV fluid, blood, plasma, tissue, a pharmaceutically-acceptable excipient, or a pharmaceutically-acceptable combination of two or more of the foregoing.
Embodiment 30. The system of any one of embodiments 27-29, wherein each container in the plurality of containers is adapted to dispense the fluid via a nipple or a cannula.
Embodiment 31. The system of any one of embodiments 27-30, wherein each container in the plurality of containers is a baby bottle or a fluid storage bag.
Embodiment 32. The system of any one of embodiments 27-31, wherein the plurality of containers comprises at least one baby bottle and at least one fluid storage bag.
Embodiment 33. The system of any one of embodiments 27-32, wherein the plurality of container tracking devices comprises at least one container tracking device of any one of embodiments 1-26.
Embodiment 34. The system of any one of embodiments 27-33, wherein the fluid comprises a substance received from the donor.
Embodiment 35. The system of embodiment 34, wherein the fluid comprises a substance received from the donor to be administered to the donor.
Embodiment 36. The system of embodiment 34, wherein the fluid comprises a substance received from the donor to be administered to the intended recipient.
Embodiment 37. The system of embodiment 36, wherein the donor is a mother, and the intended recipient is the mother's infant.
Embodiment 38. The system of embodiment 36, wherein the intended recipient is not the donor's infant.
Embodiment 39. The system of embodiment 38, wherein the donor and the recipient are pre-matched.
Embodiment 40. The system of embodiment 39, wherein the donor and the recipient are not pre-matched, and the plurality of containers are adapted to store the fluid in a fluid bank.
Embodiment 41. The system of any one of embodiments 27-40, wherein the identifying device for the donor comprises a barcode, a radio-frequency identification device, or both.
Embodiment 42. The system of embodiment 41, wherein the identifying device for the donor is configured to be semi-permanently attached to the donor.
Embodiment 43. The system of embodiment 42, wherein the identifying device for the donor is affixed to a wristband or an ankle band to be worn by the donor.
Embodiment 44. The system of any one of embodiments 27-43, wherein the identifying device for the intended recipient comprises a barcode, a radio-frequency identification device, or both.
Embodiment 45. The system of embodiment 44, wherein the identifying device for the intended recipient is configured to be semi-permanently attached to the intended recipient.
Embodiment 46. The system of embodiment 45, wherein the identifying device for the intended recipient is affixed to a wristband or an ankle band to be worn by the intended recipient.
Embodiment 47. The system of any one of embodiments 27-46, wherein the receiving computer is configured to communicate with the plurality of container tracking devices via short-wavelength UHF radio waves, a wireless local area network, infrared, a cellular network, or a combination thereof.
Embodiment 48. The system of any one of embodiments 27-47, wherein the receiving computer is chosen from a server, a desktop computer, a tablet computer, a wearable computer, and a smart phone.
Embodiment 49. The system of any one of embodiments 27-48, wherein each container tracking device in the plurality of container tracking devices is configured to retain identity data of the donor and a start time data.
Embodiment 50. The system of any one of embodiments 27-49, wherein each container tracking device in the plurality of container tracking devices is configured to be removed from a first container and reversibly coupled to a second container in the plurality of containers.
Embodiment 51. The system of embodiment 50, wherein the first container is a fluid storage bag, and the second container is a baby bottle.
Embodiment 52. A method for tracking a container comprising:
obtaining the system for tracking a plurality containers of any one of embodiments 27-51;
selecting a first container from the plurality of containers, the first container having coupled to it a first container tracking device from the plurality of container tracking devices;
registering the first container tracking device to the identifying device for a donor to obtain an identity of the donor;
receiving in the interior of the first container a fluid;
measuring, using the first container tracking device, a first exterior temperature of the first container that correlates to a temperature of the fluid in the interior of the first container;
transmitting from the first container tracking device to the receiving computer the identity of the donor and the first exterior temperature;
timing a duration of the fluid in the first container, using the first container tracking device to obtain time data and transmitting the time data to the receiving computer, or using the receiving computer to obtain time data;
periodically measuring, using the first container tracking device, subsequent exterior temperatures of the first container that correlate to subsequent temperatures of the fluid in the interior of the first container;
periodically transmitting from the first container tracking device to the receiving computer the subsequent exterior temperatures;
receiving at the receiving computer the identity of the donor, the first exterior temperature, and the subsequent exterior temperatures;
recording at the receiving computer the identity of the donor, the first exterior temperature, the subsequent exterior temperatures, and a time elapsed since the receiving of the first temperature;
optionally associating the identity of the donor with an identity of an intended recipient;
causing the first container tracking device to interrogate an identity of a potential recipient when the first container tracking device and the potential recipient are brought in close proximity;
transmitting the identity of the potential recipient from the first container tracking device to the receiving computer;
receiving at the receiving computer the identity of the potential recipient;
determining at the receiving computer whether the identity of the potential recipient matches the identity of the donor or optionally the identity of the intended recipient;
comparing at the receiving computer the subsequent exterior temperatures over the time elapsed against an acceptable temperature profile;
calculating a permission code based on the determining and the comparing;
transmitting from the receiving computer to the first container tracking device the permission code;
providing, at the first container tracking device, an alert based on the permission code.
Embodiment 53. The method of embodiment 52, wherein the selecting the first container comprises coupling to the first container the first container tracking device.
Embodiment 54. The method of any one of embodiments 52-53, wherein the registering the first container tracking device to the identifying device for the donor comprises
scanning, from the first container tracking device, the identifying device for the donor to obtain the identity of the donor; and
transmitting from the first container tracking device to the receiving computer the identity of the donor.
Embodiment 55. The method of any one of embodiments 52-54, wherein the comparing at the receiving computer the subsequent temperatures over the time elapsed comprises correlating the subsequent exterior temperatures with the subsequent temperatures of the fluid in the interior of the first container; and
comparing the subsequent temperatures of the fluid in the interior of the first container against the acceptable temperature profile.
Embodiment 56. The method of any one of embodiments 52-55, wherein the comparing at the receiving computer the subsequent temperatures over the time elapsed comprises searching for subsequent temperatures outside of a predetermined temperature range to identify one or more temperature extremes.
Embodiment 57. The method of embodiment 56, wherein the comparing at the receiving computer the subsequent temperatures over the time elapsed comprises determining whether at least one of the one or more temperature extremes exceeds an acceptable duration.
Embodiment 58. The method of any one of embodiments 52-57, further comprising retaining the identity of the first donor on the first container tracking device.
Embodiment 59. The method of any one of embodiments 52-58, further comprising retaining a start time data on the first container tracking device;
wherein the start time data corresponds to the receiving in the interior of the first container the fluid.
Embodiment 60. The method of any one of embodiments 52-59, wherein the comparing at the receiving computer the subsequent exterior temperatures over the time elapsed against an acceptable temperature profile comprises determining whether a final temperature exceeds an acceptable administration temperature.
Embodiment 61. The method of any one of embodiments 52-60, wherein the comparing at the receiving computer the subsequent exterior temperatures over the time elapsed against an acceptable temperature profile comprises determining whether the fluid has been heated above an acceptable heating temperature.
Embodiment 62. A method for managing a supply of a fluid comprising:
obtaining the system for tracking a plurality of containers of any one of embodiments 27-51;
registering the plurality of containers to a donor to establish an identity of the donor;
receiving, in one or more containers in the plurality of containers, the fluid associated with the donor;
transmitting from each of the one or more container tracking devices a signal when each container receives the fluid;
receiving at a receiving computer the signal from each of the one or more containers;
measuring a duration of the fluid in each container based on the receiving computer receiving the signal from each of the one or more container tracking devices;
calculating a permission code for each container based on the measuring the duration of the fluid in each container;
transmitting from the receiving computer the permission code for each container;
providing, at each container tracking device for the one or more containers, an alert based on the permission code.
Embodiment 63. The method of embodiment 62, further comprising:
measuring, at each container tracking device, an exterior temperature of each container that correlates to a temperature of the fluid in the interior of each container;
periodically transmitting from each container tracking device to the receiving computer the exterior temperatures of each container;
comparing at the receiving computer the exterior temperatures for each container against an acceptable temperature profile;
wherein the calculating a permission code is further based on the comparing.
Embodiment 64. The method of any one of embodiments 62-63, further comprising:
causing each container tracking device of the one or more containers to interrogate an identity of a potential recipient when each container tracking device and the potential recipient are brought in close proximity;
transmitting the identity of the potential recipient from each container tracking device to the receiving computer;
receiving at the receiving computer the identity of the potential recipient;
determining at the receiving computer whether the identity of the potential recipient matches the identity of the donor or optionally an identity of an intended recipient;
wherein the calculating a permission code is further based on the determining.
Embodiment 65. The method of any one of embodiments 62-64, wherein the calculating the permission code for each container based on the measuring the duration of the fluid in each container comprises calculating a positive permission code for the container having the longest duration.
Embodiment 66. The method of any one of embodiments 62-65, wherein the calculating the permission code for each container based on the measuring the duration of the fluid in each container comprises calculating a positive permission code for the container having the shortest duration.
Embodiment 67. The method of any one of embodiments 62-66, wherein the calculating the permission code for each container based on the measuring the duration of the fluid in each container comprises calculating a negative permission code for each container having a duration exceeding a predetermined duration.
Embodiment 68. The method of any one of embodiments 62-67, wherein the negative permission code causes the providing, at each container tracking device for the one or more containers, a permanent negative alert based on the negative permission code for each container having the duration exceeding the predetermined duration.
Embodiment 69. The method of any one of embodiments 62-68, further comprising:
retaining on the plurality of container tracking devices the identity of the donor.
Embodiment 70. The method of any one of embodiments 62-69, further comprising:
retaining, on the container tracking devices for each of the one or more containers receiving the fluid associated with the donor, a start time data corresponding to the receiving in each container the fluid.
Embodiment 71. The method of any one of embodiments 63-70, wherein the comparing at the receiving computer the exterior temperatures for each container against an acceptable temperature profile comprises determining whether a final temperature exceeds an acceptable administration temperature.
Embodiment 72. The method of any one of embodiments 63-71, wherein the comparing at the receiving computer the exterior temperatures for each container against an acceptable temperature profile comprises determining whether the fluid has been heated above an acceptable heating temperature.
Embodiment 73. The method of any one of embodiments 63-72, further comprising:
after the periodically transmitting from each container tracking device to the receiving computer the exterior temperatures of each container,
transferring the fluid from a first container among the plurality of containers to a second container;
uncoupling the first container tracking device from the first container;
reversibly coupling the first container tracking device to the second container;
periodically measuring, using the first container tracking device, subsequent exterior temperatures of the second container that correlate to a temperature of the fluid in the interior of the second container;
periodically transmitting from the first container tracking device to the receiving computer the subsequent exterior temperatures of the second container;
timing a duration of the fluid in the second container, using the first container tracking device to obtain time data and transmitting the time data to the receiving computer, or using the receiving computer to obtain the time data.
Embodiment 74. The method of embodiment 73, wherein the first container is a fluid storage bag, and the second container is a baby bottle.

As previously stated, detailed embodiments of the present invention are disclosed herein; however, it is to be understood that the disclosed embodiments are merely exemplary of the invention that may be embodied in various forms. It will be appreciated that many modifications and other variations stand within the intended scope of this invention as claimed below. Furthermore, the foregoing description of various embodiments does not necessarily imply exclusion. For example, “some” embodiments may include all or part of “other” and “further” embodiments within the scope of this invention. In addition, “a” does not mean “one and only one;” “a” can mean “one and more than one.”

1.-51. (canceled)

52. A method for tracking a container comprising:
obtaining a system for tracking a plurality of containers, comprising:
the plurality of containers, wherein each container is adapted to receive, store, and dispense a fluid;
a plurality of container tracking devices, each container tracking device in the plurality of container tracking devices being coupled to a container in the plurality of containers, each container tracking device being configured to measure an exterior temperature of the container that correlates to a temperature of a fluid in the interior of the container to obtain temperature data, a proximity sensor configured to interrogate an identity of a donor or potential recipient when the container tracking device and the donor or potential recipient are brought in close proximity to obtain identity data, and a timer configured to time a duration of the fluid in the interior of the container to obtain time data;
an identifying device for a donor configured to be interrogated by the proximity sensor of each container tracking device in the plurality of container tracking devices;
optionally, an identifying device for an intended recipient, the identifying device for the intended recipient being configured to be interrogated by the proximity sensor of each container tracking device in the plurality of container tracking devices;
a receiving computer configured
to receive the temperature data, the identity data, and the time data from each container tracking device in the plurality of container tracking devices,
to calculate permission codes based on the temperature data, the identity data, and the time data, and
to selectively transmit the permission codes to the container tracking devices in the plurality of container tracking devices,
wherein each container tracking device is further configured to selectively receive the permission codes, and provide alerts based on the permission codes;
selecting a first container from the plurality of containers, the first container having coupled to it a first container tracking device from the plurality of container tracking devices;
registering the first container tracking device to the identifying device for a donor to obtain an identity of the donor;
receiving in the interior of the first container a fluid;
measuring, using the first container tracking device, a first exterior temperature of the first container that correlates to a temperature of the fluid in the interior of the first container;
transmitting from the first container tracking device to the receiving computer the identity of the donor and the first exterior temperature;
timing a duration of the fluid in the first container, using the first container tracking device to obtain time data and transmitting the time data to the receiving computer, or using the receiving computer to obtain time data;
periodically measuring, using the first container tracking device, subsequent exterior temperatures of the first container that correlate to subsequent temperatures of the fluid in the interior of the first container;
periodically transmitting from the first container tracking device to the receiving computer the subsequent exterior temperatures;
receiving at the receiving computer the identity of the donor, the first exterior temperature, and the subsequent exterior temperatures;
recording at the receiving computer the identity of the donor, the first exterior temperature, the subsequent exterior temperatures, and a time elapsed since the receiving of the first temperature;
optionally associating the identity of the donor with an identity of an intended recipient;
causing the first container tracking device to interrogate an identity of a potential recipient when the first container tracking device and the potential recipient are brought in close proximity;
transmitting the identity of the potential recipient from the first container tracking device to the receiving computer;
receiving at the receiving computer the identity of the potential recipient;
determining at the receiving computer whether the identity of the potential recipient matches the identity of the donor or optionally the identity of the intended recipient;
comparing at the receiving computer the subsequent exterior temperatures over the time elapsed against an acceptable temperature profile;
calculating a permission code based on the determining and the comparing;
transmitting from the receiving computer to the first container tracking device the permission code;
providing, at the first container tracking device, an alert based on the permission code.

53. The method of claim 52, wherein the selecting the first container comprises coupling to the first container the first container tracking device.

54. The method of claim 52, wherein the registering the first container tracking device to the identifying device for the donor comprises
scanning, from the first container tracking device, the identifying device for the donor to obtain the identity of the donor; and
transmitting from the first container tracking device to the receiving computer the identity of the donor.

55. The method of claim 52, wherein the comparing at the receiving computer the subsequent temperatures over the time elapsed comprises
correlating the subsequent exterior temperatures with the subsequent temperatures of the fluid in the interior of the first container; and
comparing the subsequent temperatures of the fluid in the interior of the first container against the acceptable temperature profile.

56. The method of claim 52, wherein the comparing at the receiving computer the subsequent temperatures over the time elapsed comprises searching for subsequent temperatures outside of a predetermined temperature range to identify one or more temperature extremes.

57. The method of claim 56, wherein the comparing at the receiving computer the subsequent temperatures over the time elapsed comprises determining whether at least one of the one or more temperature extremes exceeds an acceptable duration.

58. The method of claim 52, further comprising retaining the identity of the first donor on the first container tracking device.

59. The method of claim 52, further comprising retaining a start time data on the first container tracking device;
wherein the start time data corresponds to the receiving in the interior of the first container the fluid.

60. The method of claim 52, wherein the comparing at the receiving computer the subsequent exterior temperatures over the time elapsed against an acceptable temperature profile comprises determining whether a final temperature exceeds an acceptable administration temperature.

61. The method of claim 52, wherein the comparing at the receiving computer the subsequent exterior temperatures over the time elapsed against an acceptable temperature profile comprises determining whether the fluid has been heated above an acceptable heating temperature.

62.-74. (canceled)
